(19) United States
(12) Patent Application Publication
(10) Pub. No.: US 2026/0013838 A1
(43) Pub. Date: Jan. 15, 2026

(54) FECAL SPECIMEN COLLECTION CONTAINER AND METHODS OF PROCESSING FECAL SAMPLES

(71) Applicant: EPITOPE DIAGNOSTICS, INC., San Diego, CA (US)

(72) Inventor: Ping Gao, San Diego, CA (US)

(21) Appl. No.: 19/269,714

(22) Filed: Jul. 15, 2025

Related U.S. Application Data
(60) Provisional application No. 63/671,441, filed on Jul. 15, 2024.

(51) Int. Cl. A61B 10/00

(52) U.S. Cl. CPC A61B 10/0038

(57) ABSTRACT

A fecal specimen collection container, having an upper chamber positioned above a lower chamber, the upper chamber including a wiper and a slot that accesses the lower chamber; a spatula having a length greater than that of the upper chamber and configured to traverse the slot to access the lower chamber, wherein the wiper is configured to wipe the spatula when traversing the slot; and a lid configured to cap the upper chamber, the lid having a reversibly closable access port to access the upper chamber when capped.

### CROSS REFERENCE TO RELATED APPLICATIONS

This application claims priority to U.S. provisional patent application No. 63/671,441, filed Jul. 15, 2024.

### TECHNICAL FIELD

The invention relates to devices, kits, and methods for collecting and processing biological specimens and more specifically to a fecal specimen collection container and methods of collecting and processing fecal specimens using the container.

### BACKGROUND OF THE INVENTION

Biological specimens are routinely collected and tested to assess the health of the subject; however, some specimens are easier to collect and process for testing than others. For example, test tubes preloaded with anticoagulants and stored under vacuum are commonly used for blood collection. A phlebotomist uses a double-sided needle to pierce the tube at one end, and the patient's vein at the other end. The vacuum automatically draws blood into the tube, where it is mixed with the anticoagulant. The testing facility then simply retrieves the liquid sample from the container. As another example, urine samples are often collected by the subject urinating in a specimen cup. Then the testing facility simply pipettes or pours the liquid sample from the cup for testing.

Collecting fecal specimens and processing fecal samples presents unique challenges compared to liquid samples, at least in part because feces itself can vary significantly in firmness and consistency. That is, fecal collection techniques must be useful for both firm and highly soft specimens. Moreover, in contrast to blood collection, fecal specimens are typically collected by the subject in need of testing or a family member, not professionals, and therefore should require minimal skill. Moreover, the collection of feces often adds additional stress to the subject or family member.

Fecal specimen collection often uses a spatula and a specimen cup to scoop and house the feces. The specimen cup is then provided to the testing facility, where the feces is further sampled (secondary collection). This secondary fecal sample is then suspended, dissolved, and submitted to liquid-based extraction techniques for testing. For example, in order to quantitatively measure fecal calprotectin, a stool specimen is collected by a patient using a plastic tube or plastic container with a single chamber to house the stool. Then this container is delivered to a professional laboratory. Secondary collection of the stool sample is then performed by laboratory technicians using a quantitative stool sample collection device (QSSCD), such as a Q.S.E.T from DiaSorin, Fecal Sample Collection Tube from Immundiagnostik (IDK Extract, SKU K6999.US), Bulhmann (CALEX, Cat #B-CALEX-C), Epitotope Diagnostics (Cat # 30210, KT-843), etc. This secondary collection is then used as the basis for the calprotectin assay.

Since secondary collection and stool-based assays are becoming standardized, the initial collection containers themselves must conform to certain tolerances. For example, since the technician must reach into the primary collection container with the collection wand or stick of the QSSCD, the fecal specimen should be stored in a primary collection chamber that is shorter than the collection wand or stick of the QSSCD, which means the stool container is usually no more than 7 cm in height. One implication is that since the specimen collecting spatula (blade+handle) used by the patient is typically stored in the collection container, it must be shorter than the height of the stool container. Thus, on the one hand, following the above standardized practices encourages shortening the spatula; but on the other hand, patient stress increases as the length of the spatula decreases, especially when multiple scoops are needed. For example, while it may initially be preferred to integrate the spatula with the container, due to size constraints a stool container with integrated spatula usually only collects 1 gram or less stool in a single scoop, thereby forcing the subject to scoop then wipe the spatula multiple times to obtain a sufficient volume for downstream testing.

In this regard, reducing stress and improving compliance may be accomplished if a longer spatula (blade+handle) could be used. One option to increase spatula size is to use a separate free standing long spoon as a collecting spatula. However, even with a long spoon it is difficult to off load soft feces into the container without a wiping process. Scraping with multiple utensils (e.g. two spoons) would lead to multiple tools being exposed to feces. This would be unpleasant and increase stress, as well as one must throw the contaminated spoon(s) into a trash bin at home, which is again not a pleasant maneuver. Presumably one could scrape the spatula against the opening of the specimen container to at least minimize the number of utensils to be discarded, but this could cause a further mess when capping the container, lodge the feces at the top of the container, and still does not avoid problems associated with discarding this large spatula.

Therefore, there remains a need to develop an approach where the spatula can be stored within the specimen collection container to avoid discarding, yet also be easily used to collect a larger amount of feces for testing.

However, even after hypothetically solving the above, there remain additional challenges. Again, once the specimen collection container reaches the testing facility, the feces is further sampled (secondary collection), and is suspended, dissolved, and extracted in a liquid solution manually by a lab technician or potentially using automation. Because of the variability often encountered in the physical characteristics of feces, from very hard to very soft with different stickiness, in many instances, the specimen sticks to the spatula and is therefore difficult to offload by the technician. Like at home, scraping the feces from the spatula by the test facility requires the use of additional tools; however, in a laboratory setting, these are considered biohazardous waste and therefore must be disposed of under heightened requirements. In addition, the specimen container remains uncapped while the container and tools are manipulated, which releases undesirable odors.

Accordingly, in addition to the need to develop an approach where the spatula can be stored within the specimen collection container and which can be used to easily collect a variable amount of feces for testing, there also remains a need for a configuration to improve subsequent secondary collection or sampling by technicians.

### SUMMARY OF THE INVENTION

The invention addresses the above-described problems and provides related benefits. In particular, the specimen collection containers and methods discussed herein are suitable for use with QSSCD laboratory practices while also improving the specimen collection experience for the patient and lab technicians. In addition, the collection containers and procedures described herein will ease the transition to automated sampling and testing of fecal specimens. The above is achieved in one aspect of the invention by way of a fecal specimen collection container, which includes a body, which includes an upper chamber positioned above a lower chamber, where the upper chamber is configured to house feces and includes a wiper and a slot that accesses the lower chamber; a spatula (blade+handle) having a length greater than that of the upper chamber and configured to traverse the slot to access the lower chamber, and where the wiper is configured to wipe the spatula when traversing the slot; and a lid configured to cap the upper chamber, the lid having a reversibly closable access port to access the upper chamber when capped.

The height of the container's upper chamber should less than 15 centimeters. In a preferred embodiment, the height of the upper chamber is 5-8 centimeters. In any event, the height of the upper chamber is most preferably less than the length of a QSSCD collection wand/stick if used in subsequent processing.

Preferably, the container is suitable for use in an automated sample processor or system, which performs quantitative stool sample collection via a quantitative stool sample collection device (QSSCD).

In some embodiments, when fully inserted into the slot, an upper edge of the blade of the spatula is at the same height or is preferably slightly higher in position than the wiper so that a lip portion of the spatula blade abuts the wiper, thereby preventing or reducing potential spilling of the upper chamber contents into the lower chamber.

In some embodiments, when the spatula is fully inserted through the slot and when the upper chamber has a concave floor with inwardly tapered wall, a top region of the inwardly tapered wall aligns with the top the spatula blade, such as the top of the lip in some instances. In any of the above, after collection of feces, the spatula remains within the specimen collection container, which prevents an unpleasant discharge experience.

In some embodiments, the blade of the spatula is capable to collect up to 10 grams of feces per scoop. In a preferred embodiment, the blade of the spatula collects 2-5 grams of feces per scoop. As the spatula traverses the slot to reach the lower chamber, the feces is scraped off from the blade and is offloaded to the floor of the upper chamber. Among the benefits of configurations described throughout this document is that if needed, one may perform multiple collections using the same spatula without added stress due to the increased spatula size and efficient wiping configurations. Collection can be repeated multiple times until the desired amount of feces is collected. As guidance, typically 3 grams of feces will fill the bottom of the upper chamber to a height of at least 1.5 centimeters; however this can vary.

The floor of the upper chamber can be contoured to improve collection of feces with different consistencies. That is, while the floor can be planar, in some embodiments, the upper chamber has a concave floor, which would include, in essence, a generally inwardly taper wall to feed feces to the lowermost region or vertex region. Thus, the concave floor can include a substantially uniform tapered wall akin to an inverted partial or truncated cone, can include a generally parabolic curved wall, can include a stepwise structure, or any other configuration directing offloaded feces to the lowermost portion of the floor (e.g vertex region). A concave floor can also have a plurality of depressions or recesses at its vertex region for still further solids collection.

In some embodiments, the concave floor has a vertex region that is directly beneath the access port of the lid, but in other embodiments, the vertex region is not directly beneath the access port of the lid.

In preferred embodiments, the concave floor can have a cutaway that lowers or recesses the slot into the inwardly tapered wall and thus closer to the lowest region of the upper chamber. This configuration is particularly useful when the top of the spatula blade is configured with a lip, so that the lip presses against or covers the wiper when the spatula is fully inserted through the slot to prevent or reduce spilling of the specimen into the lower chamber during shipment or manipulation of the container. Most preferably, in such embodiments, when the spatula is fully inserted through the slot, a top of the wall substantially aligns or is substantially planar with the top lip of blade.

In some embodiments, the wiper forms part of an edge of the slot.

In preferred embodiments, the lower chamber has substantially a same shape as the blade of the spatula (but slightly larger) to form a similarly shaped sleeve for insertion of the blade, thereby minimizing the volume of the lower chamber. Most preferably, both are generally concave so that the contours follow one another. Most preferably, when inserted within the lower chamber, the blade fills more than 80% of the volume of the lower chamber.

In some embodiments, the access port of the lid is reversibly closable via a slide that reversibly slides over the access port. In some embodiments, the slide has an ascending ridge. In some embodiments, the lid has an ascending ridge adjacent the ascending ridge of the slide when the access port is open. In some embodiments, the lid has a flange forming at least part of a track over which the slide moves between an open position and a closed position to open and close the access port. In some embodiments, straight downward from the access port is the lowest portion of the upper chamber of the body. In another embodiment, the access port of the lid is reversibly closable via a flip-up and flip-down/snap maneuver.

In a related aspect of the invention, a kit for the collection of a fecal specimen is provided, the kit including any one of the fecal specimen collection containers described throughout this document; a sealable collection bag configured to house the collection container, the collection bag containing an absorbent pad configured to absorb fluid; and a fecal specimen catcher, which includes a sleeve configured for attachment to a toilet seat for spanning and hanging below the toilet seat for catching feces. The kit can also include one or more of a mailer configured to house the collection bag with collection container, and instructions for fecal specimen collection.

Preferably, the fecal specimen catcher is made of paper and wraps around the toilet seat. The paper can be a continuous loop or can form loops at opposing ends. The catcher can also include a water-soluble film (e.g. polyvinyl alcohol) and a water-soluble adhesive. Most preferably, the catcher is biodegradable and flushable.

In another related aspect, a method of processing a fecal specimen is provided, which includes providing any one of the fecal specimen collection containers or kits described herein; loading the spatula with feces; inserting the spatula through the slot while closing the lid so that the wiper wipes the feces from the spatula and into the upper chamber as the lid is capped; opening the access port; and retrieving a fecal sample from the upper chamber.

### BRIEF DESCRIPTION OF DRAWINGS

Embodiments of the invention can be better understood with reference to the following drawings, which are part of the specification and represent preferred embodiments. The components in the drawings are not necessarily to scale, emphasis instead being placed upon illustrating the principles of the invention. And, in the drawings, like reference numerals designate corresponding parts throughout the several views.

FIG. 1 is an exemplary embodiment of a fecal specimen collection container 100 having a planar floor 25 in the upper chamber 14a with a slot 16 having an integrated wiper 12 for removing feces from the spatula 10.

FIG. 2 is a cutaway view of another exemplary embodiment of a fecal specimen collection container 200 having a concave floor 26 with inwardly tapered wall 27 for directed wiped feces to a vertex region 30 and also shows preferred complementary shaping between a spatula blade 24 and a sleeve 32 to minimize the volume within the lower chamber 14b/sleeve 32.

FIG. 3A depicts the exemplary spatula 10 with blade 24 from FIG. 2 more clearly depicting a lip 22. FIG. 3B shows an exemplary arced contour of a sleeve 32 and shows an exemplary vertex region 30 for receiving the wiped specimen.

FIG. 4 depicts an exemplary lid 18 and with an exemplary integrated spatula 10 better showing the access port 20, which is accessed via a slide 34 that moves along a flange 40.

FIG. 5 depicts a kit 50 for the collection of a fecal specimen.

### DETAILED DESCRIPTION

This disclosure is directed to fecal specimen collection containers and their use in the processing of fecal specimens. The containers provide an easy to use spatula for the collection of feces by the subject or patient, and a wiper configuration that wipes the spatula clean as it is inserted into the body of the device, which easily allows the user to provide as much specimen as required. The container is also configured with optional structural features that make it ideal for use in automated sample testing systems.

For clarity of disclosure, and not by way of limitation, the invention is discussed according to different detailed embodiments; however, the skilled artisan would recognize that features of one embodiment can be combined with other embodiments and is therefore within the intended scope of the invention.

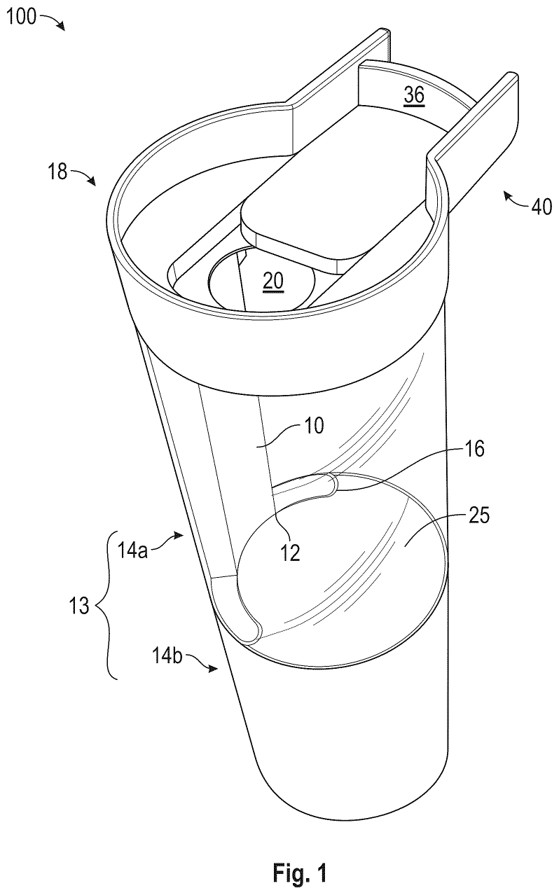

Beginning with FIGS. 1-4 collectively, what is disclosed is a fecal specimen collection container 100, 200 having an body 13 with an upper chamber 14a, which is configured to house feces and which is preferably formed from a substantially optically transparent material (e.g. transparent plastic), positioned above a lower chamber 14b; and a spatula 10 having a length greater than that of the upper chamber 14a and configured to access the lower chamber 14b by passing through a slot 16; during which feces is wiped from the spatula 10 for collection in the upper chamber 14a. A lid 18 that caps the upper chamber 14a includes a reversibly closeable access port 20 to access the upper chamber 14a for sample collection when capped.

While in some embodiments, the wiper 12 can be provided as a separate structure such as a bendable rubber blade affixed to the upper chamber 14a and overhanging the slot 16, or a raised rigid lip, FIG. 1 shows an exemplary fecal sample collection container 100, where the upper chamber 14a has a slot 16 within the floor 25, which due to its tight tolerances forms an effective wiper 12. That is, by following the contour of the spatula blade 24 under tight tolerances (e.g. a few millimeters or less), the edge(s) of the slot 16 can form the wiper 12 to wipe away feces from the spatula blade 24, as it passes through the slot 16, thereby offloading feces almost exclusively into the upper chamber 14a while storing the spatula blade 24 mostly in the lower chamber 14b. Providing the wiper 12 as part of the slot edge minimizes manufacturing costs. In various embodiments, the gap between the slot edge and spatula blade 24 can vary, however spacing of about 0.1 mm to 3 mm is preferred in that on the one hand this range tends to efficiently wipe feces from the spatula 10, yet on the other hand, the spatula 10 can be easily maneuvered into the slot 16.

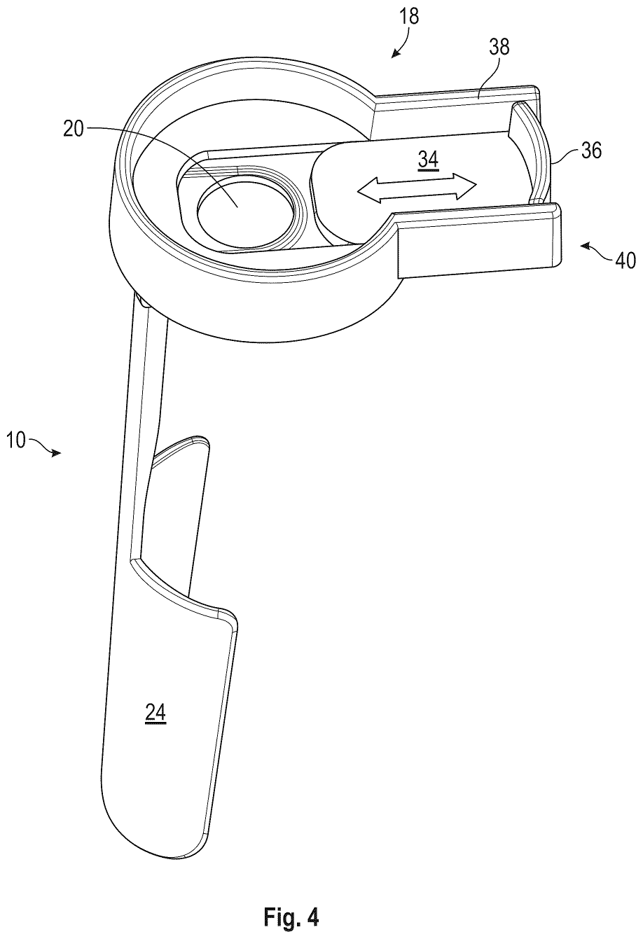

It has also been found that when integrating the spatula 10 into the lid 18 (see also FIG. 4), the spatula 10 can be more easily fitted into the slot 16 when including additional structural features that encourage its proper alignment towards the slot 16. In one configuration, the lid 18 and upper chamber 14a have complementary mating surfaces that encourage relative positioning of one to the other. As an example, by providing complementary ridge(s) and/or grooves on mating surfaces of the lid 18 and upper chamber 14a, the alignment of the spatula 10 can be biased towards the slot 16. Preferably, the lid 18 and upper chamber 14a are fitted by a snap connection that is fluid-tight.

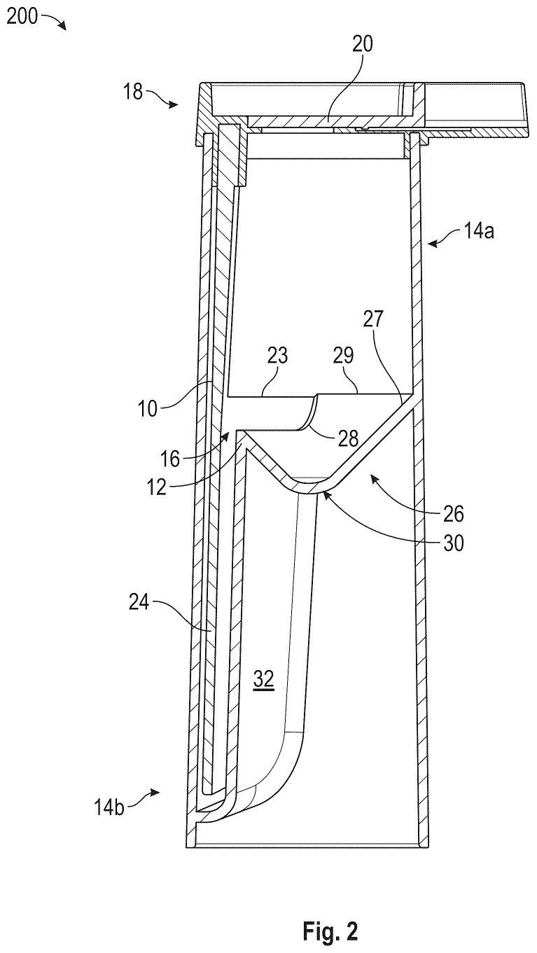

Shown better in FIG. 2, other structural modifications have also been developed in various embodiments, which still further improve the processing of fecal specimens. In some embodiments, the upper chamber 14a has been modified to provide a concave floor 26 compared to the more planar floor 25 shown in FIG. 1, which encourages the fecal specimen to accumulate at the lowermost region, which is referred to as the vertex region 30 of the concave floor 26. By “concave floor” it is meant that there is a generally inwardly tapered wall 27 to feed feces to the lowermost region or vertex region 30, whether the taper is akin to an inverted cone, an inverted partial cone, has a more curved contour, or otherwise. A benefit of this configuration is that the specimen tends to fall and accumulate into the smaller vertex region 30 after wiping, thereby making its subsequent collection easier on the one hand and reducing the likelihood of leaking into the lower chamber 14b on the other, as the slot 16 is higher and remote from the vertex region 30. Thus, subsequent secondary collection or sampling of collected feces from the upper chamber 14a can include lowering the collection/sampling device (e.g. QSSCD collection wand) towards the vertex region 30 to ensure capture. This collection/sampling depth can be preprogrammed into automation using cartesian coordinates according to the particular collection/sampling instrument or can operate by measuring resistance to penetration. Thus, preferably, the vertex region 30 of the concave floor 26 is directly below the access aperture 20, but in less preferred embodiments it is not.

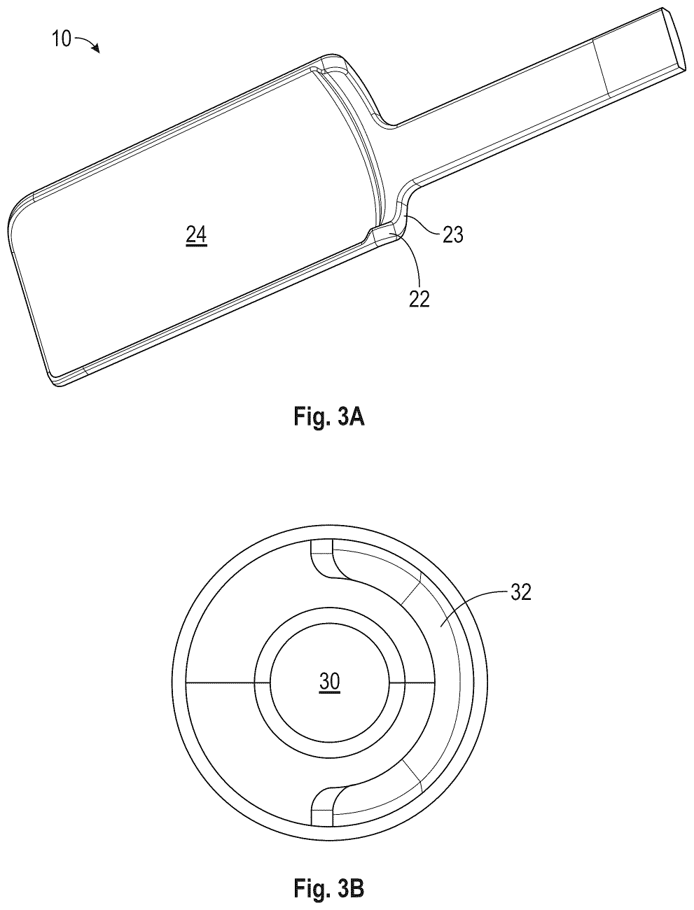

Still further embodiments of the concave floor 26 have been developed to provide improved specimen collection. In particular, a concave floor 26 has been further modified to provide a cutaway 28 along the inwardly tapered wall 27 for improved receiving of the spatula blade 24 through the slot 16. As seen best in FIG. 2, this cutaway 28 lowers the slot 16 into the wall 27. This configuration permits the floor 26 to retain the concave (e.g. inverted partial cone-like) shape and thus encourage the accumulation of feces at the vertex region 30 after wiping. However, it also improves alignment when inserting the spatula blade 24 into the slot 16 in that cutaway 28 can help capture the blade 24. Furthermore, as shown best in FIGS. 2-3A, when providing a spatula blade 24 with a lip 22, this lip 22 can press against (e.g. abut or cover) the edge of the slot 16 or wiper 14 to better seal the lower chamber 14b from the upper chamber 14a. In preferred embodiments, when the spatula 10 is fully inserted through the slot 16, the top 29 of the wall 27 substantially aligns with the top 23 of the blade 24 forming part of the spatula 10 (see FIG. 2), thereby further improving the sealing of the slot 16.

Turning back to FIGS. 1-2, since the primary function of the lower chamber 14b is to store the spatula blade 24 after wiping, the dimensions and contours of the lower chamber 14b can vary significantly. For example, in some embodiments manufacturing costs are minimized by providing a simple cylindrical lower chamber 14b (e.g. FIG. 1), but in other embodiments, the lower chamber 14b is contoured to provide a sleeve 32 for the spatula blade 34 (FIG. 2). This sleeve-like configuration can best be seen in FIG. 2 and FIG. 3B, where the lower chamber 14b is configured as a sleeve 32 that is shaped substantially the same as the spatula blade 24 but hollowed to essentially holster the blade 24, thereby significantly reducing the effective volume of the lower chamber 14b. In such embodiments, the blade 24 can fill 80% or more of the volume of the lower chamber 14b/sleeve 32. In other embodiments, the blade 24 fills 85% or more, 90% or more, or 95% or more of the lower chamber 14b/sleeve 32. When sleeved, spacing behind, in front of, and to the sides of the blade, preferably, range from 0.1 mm to 3 mm. Thus, in each embodiment, the blade 24 preferably fills less than 100% of the volume of the lower chamber 14b/sleeve 32 to ease its removal and insertion during use.

Moving on to the lid 18, each of the specimen containers 100, 200 provided herein typically includes a lid 18 that caps the upper chamber 14a, preferably, using a fluid-tight, snap fit. The lid not only provides fluid tight closure of the upper chamber 14a to prevent spilling or leaking but also provides the access port 20 for retrieving a fecal sample by the clinical worker or automation without removing the entire lid 18, thereby limiting exposure to offensive odors. Numerous features have been developed to further improve the lid 18 and access to the upper chamber 14a without its removal.

In some embodiments, the access port 20 is accessed by a flip top that flips open and snaps shut. While this operation can be performed using the clinical worker's fingers there are additional challenges imposed when automating sample collection. To improve configurations for automated processing of specimens, a slide configuration has been developed, where a slide 34 is able to slide back and forth to reversibly cover and uncover the access port 20. The slide 34 can be sealed by encircling the access port 20 with a ridge or an O-ring.

However, sliding small surfaces over one another can also be a challenge during automation. This challenge has been overcome by forming an ascending ridge 36 on the slide 34. This ascending ridge 36 provides a surface against which automation (e.g. robotic arm/finger) can push and/or pull, thereby closing and opening or opening and closing the access port 20. In some embodiments, the lid 18 also includes an ascending ridge 38 that is aligned adjacent the ascending ridge 36 of the slide 34 when the access port 20 is open and/or closed.

The slide configuration has been further improved by forming a flange 40 integral with the lid 18. The addition of a flange 40 provides numerous benefits. First, the flange 40 can act as a track for the slide 34. This permits the diameter or perimeter and thus volume of the upper chamber 14a to be reduced, which economizes on space. A second benefit is that the flange 40 improves alignment during automation. In particular, it has also been found that the flange 40 provides an asymmetric shape, which can be exploited when loading the fecal specimen collection container 100, 200 into automation, such as for sample collection and analysis. Thirdly, testing has revealed that by adding a flange 40, a lid 18 with integrated spatula 10 is easier to manipulate with the subject's fingers and thus the specimen loading experience has been improved.

Turning now to operation of the container 100, 200, a fecal specimen can be processed using any of the above-referenced fecal specimen containers 100, 200. First, feces is scooped using the spatula 10. The spatula 10 typically has a concave blade 24 to facilitate scooping of solid, semi-solid and still more liquid specimens. Preferably, the spatula is configured to load at least 2-5 grams of feces or about 10 grams of feces per scoop. The spatula 10 loaded with feces is added to the upper chamber 14a so that the spatula blade 24 traverses the slot 16 to enter the lower chamber 14b. Meanwhile, the wiper 12 ensures the fecal specimen is substantially maintained in the upper chamber 14a by wiping the feces from the spatula 10 before entry into the lower chamber 14b. This process can be repeated as needed. The lid 18, which is preferably integrated with the spatula 10, finally caps the upper chamber 14a. Once capped, the specimen container 100, 200 can be transported to any desired facility for sample testing.

Upon receipt by the testing facility, the fecal specimen container 100, 200 is preferably set upright so that any specimen tends to accumulate at the floor of the upper chamber 14a. Specimen accumulation is best when using a concave floor having vertex region 30 directly below the access port 20. The specimen container 100, 200 can be loaded into an automated system or can be further processed by hand. In either case, the access port 20 is opened and a sample retrieved by inserting a collecting or sampling structure (e.g. QSSCD collection wand). The sample is then further processed according to the particular assay.

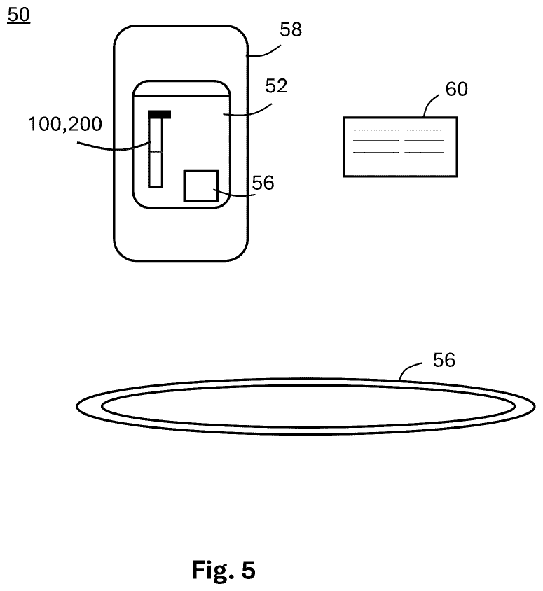

Still further improvements have been developed to improve the user's experience with the collection of a fecal specimen. In particular, any of the containers 100, 200 described above can be included in a kit 50 (see FIG. 5), which can also include a sealable collection bag 52 configured to house the collection container 100, 200, and a fecal specimen catcher 54 configured for attachment to a toilet seat for spanning and hanging below the toilet seat for catching feces.

First with respect to the collection bag 52, preferably it seals the container 100, 200, fluid tight to avoid leaking. Housed within the bag 52 is preferably an absorbent pad 56 to absorb any leaks that could potentially occur from the container 100, 200 through shipping as a precautionary step.

The catcher 54 is preferably made of a biodegradable and flushable material, such as a suitable paper. Examples include various tissue papers. The catcher 54 can also include a thin soluble film, such as a polyvinyl alchohol (PVA) film and optionally a water soluble adhesive. While the catcher 54 attaches to the toilet seat and thus spans the seat, it should also be raised from the water to avoid tearing even under weight of feces. Examples of suitable approaches include a continuous oval shaped sleeve that inserts the seat within the oval or an extended sleeve with loops at opposing ends for looping the seat.

Those having ordinary skill in the art to which the invention belongs would recognize that the kit could also include a mailer for mailing the bag 52/container 100, 200 and instructions 60 for use.

The invention described herein may be embodied in other specific forms without departing from the spirit or essential characteristics thereof. The specific embodiments previously described are therefore to be considered as illustrative of, and not limiting, the scope of the invention.

1. A fecal specimen collection container, comprising:
a body comprising an upper chamber configured to house feces, the upper chamber positioned above a lower chamber, the upper chamber comprising a wiper and a slot that accesses the lower chamber;
a spatula having a length greater than a height of the upper chamber and a concave blade configured to traverse the slot to access the lower chamber, wherein the wiper is configured to wipe the blade of the spatula when traversing the slot, further wherein the lower chamber has substantially a same shape as the blade of the spatula to form a sleeve for insertion of the blade; and
a lid configured to cap the upper chamber, the lid comprising a reversibly closable access port to access the upper chamber when capped.

2. The fecal specimen collection container of claim 1, wherein the upper chamber is less than 15 centimeters in height, preferably 5 to 8 cm in height.

3. The fecal specimen collection container of claim 1, wherein the upper chamber comprises an inwardly tapered wall forming part of a concave floor.

4. The fecal specimen collection container of claim 3, wherein the concave floor comprises a vertex region that is directly under the access port of the lid when capped.

5. The fecal specimen collection container of claim 3, wherein when the blade of the spatula is fully inserted through the slot, a top of the wall substantially aligns with a top of the blade.

6. The fecal specimen collection container of claim 3, wherein the wall comprises a cutaway that lowers the slot into the wall.

7. The fecal specimen collection container of claim 1, wherein the wiper forms part of an edge of the slot.

8. (canceled)

9. (canceled)

10. The fecal specimen container of claim 1, wherein the blade comprises a lip that abuts the wiper when the spatula is fully inserted into the body, thereby blocking access to the lower chamber through the slot.

11. The fecal specimen container of claim 1, wherein the blade fills more than 80% the lower chamber's volume when fully inserted.

12. The fecal specimen collection container of claim 1, wherein the spatula is configured to collect at least 2-5 grams of feces, optionally 10 grams of feces, per scoop.

13. The fecal specimen collection container of claim 1, wherein the spatula is joined to the lid.

14. The fecal specimen collection container of claim 1, wherein the lid snaps on fluid tight to the body.

15. A fecal specimen collection container, comprising:
a body comprising an upper chamber configured to house feces, the upper chamber positioned above a lower chamber, the upper chamber comprising a wiper and a slot that accesses the lower chamber;
a spatula having a length greater than a height of the upper chamber and configured to traverse the slot to access the lower chamber, wherein the wiper is configured to wipe the spatula when traversing the slot; and
a lid configured to cap the upper chamber, the lid comprising a reversibly closable access port to access the upper chamber when capped, wherein the access port is reversibly closable fluid tight via a slide that reversibly slides over the access port.

16. The fecal specimen collection container of claim 15, wherein the slide comprises an ascending ridge.

17. The fecal specimen collection container of claim 16, wherein the lid comprises an ascending ridge adjacent to the ascending ridge of the slide when the access port is fully open.

18. The fecal specimen collection container of claim 15, wherein the lid comprises a flange forming at least part of a track over which the slide moves between an open position and a closed position to open and close the access port.

19. A kit for the collection of a fecal specimen, the kit comprising:
a) the fecal specimen collection container of claim 1;
b) a sealable collection bag configured to house the collection container, the collection bag containing an absorbent pad configured to absorb fluid; and
c) a fecal specimen catcher comprising a sleeve configured for attachment to a toilet seat for spanning and hanging below the toilet seat for catching feces;
d) optionally a mailer configured to house the collection bag with collection container; and
e) optionally instructions for fecal specimen collection.

20. The kit of claim 19, wherein the fecal specimen catcher is made of paper and optionally a water-soluble film (e.g. polyvinyl alcohol), and optionally a water-soluble adhesive.

21. A method of processing a fecal specimen, comprising:
a) providing the fecal specimen collection container of claim 1;
b) loading the spatula with feces;
c) inserting the spatula through the slot while closing the lid so that the wiper wipes the feces from the spatula and into the upper chamber as the lid is capped;
d) opening the access port; and
e) retrieving a fecal sample from the upper chamber.

22. The method of claim 21, wherein the lid of the container comprises a slide that reversibly slides over the access port and the slide comprises an ascending ridge, further wherein the step of opening the access port comprises pushing or pulling against the ascending ridge using a robotic arm.
